(19) United States
(12) Patent Application Publication
(10) Pub. No.: US 2026/0338896 A1
(43) Pub. Date: Oct. 29, 2026

(54) METHOD FOR DETERMINING AN OPTIMAL INVERSION TIME FOR AN "INVERSION RECOVERY" RADIO FREQUENCY PULSE SEQUENCE OF A MAGNETIC RESONANCE FOR ACQUIRING LATE IMAGES AFTER ADMINISTERING A PARAMAGNETIC CONTRAST MEDIUM

(71) Applicants: UNIVERSITÀ DEGLI STUDI DI MILANO – BICOCCA, Milano (IT); ISTITUTO AUXOLOGICO ITALIANO, Milano (IT); UNIVERSITÀ CA' FOSCARI VENEZIA, Venezia (IT)

(72) Inventors: Daniela BESOZZI, Milano (IT); Marco NOBILE, Venezia (IT); Daniele PAPETTI, Milano (IT); Camilla TORLASCO, Milano (IT)

(73) Assignees: UNIVERSITÀ DEGLI STUDI DI MILANO – BICOCCA, Milano (IT); ISTITUTO AUXOLOGICO ITALIANO, Milano (IT); UNIVERSITÀ CA' FOSCARI VENEZIA, Venezia (IT)

(21) Appl. No.: 19/106,033

(22) PCT Filed: Aug. 28, 2023
(86) PCT No.: PCT/EP2023/073475

§ 371 Date: Feb. 24, 2025

(30) Foreign Application Priority Data
Aug. 31, 2022 (IT) 102022000017907

(51) Int. Cl. G01R 33/54; A61B 5/00; A61B 5/055; G01R 33/56; G06N 20/20; G16H 50/20

(52) U.S. Cl. CPC G01R 33/543; A61B 5/0044; A61B 5/055; G01R 33/5601; G01R 33/5602; G01R 33/5608; G06N 20/20; G16H 50/20

(57) ABSTRACT

A method for determining an optimal inversion time for an “Inversion Recovery” radio frequency pulse sequence of a magnetic resonance for acquiring late images after administering a paramagnetic contrast medium by a control and image capture device associated with a magnet structure of a magnetic resonance apparatus and connected to a computer adapted to exchange information and instructions with the control and image capture device is described, the method including a training sequence including optimizing a machine learning model starting from a plurality of calculated optimal inversion time values correlated to significant data of a related examined subject and specific parameters of a carried-out examination as established in the course of a plurality of sample assessments; and a calculation sequence including a first step wherein significant data of a subject to be examined and specific parameters of an examination carried out by the magnetic resonance apparatus are collected.

### TECHNICAL FIELD

The present invention refers to a method for determining the optimal inversion time for an “Inversion Recovery” radio frequency pulse sequence to be used in a magnetic resonance for acquiring late images after administering a paramagnetic contrast medium, in a so-called late imaging.

The invention particularly, but not exclusively, refers to a method for determining the optimal inversion time for an “Inversion Recovery” radio frequency pulse sequence to be used in a magnetic resonance for acquiring late images of an area of interest, in particular of the myocardium of a subject to be examined, after administering a paramagnetic contrast medium and the following description is made with reference to this field of application with the sole purpose of simplifying the exposition thereof.

### BACKGROUND ART

As it is well known, the magnetic resonance is a diagnostic technique based on the application of a high intensity magnetic field and radio frequency pulse sequences to an area of the body of a subject to be examined.

Said method is widely used in the diagnostic field since it is minimally invasive, it does not require the use of ionizing radiations and allows obtaining images with an extremely high anatomical detail, possibly integrated with functional information.

Essentially, the magnetic resonance technique uses a static magnetic field (BO) generated by the magnetic resonance apparatus. In particular, the subject to be examined is positioned inside said apparatus and is thus exposed to the static magnetic field BO. Radio frequency pulses, emitted in sequence, called radio frequency pulse sequences, are superimposed to said static magnetic field BO. More particularly, the features of the used pulse sequence, such as the time which passes between a pulse and the next one, the time which passes after a pulse for detecting a signal, the duration of the pulse, the direction of the pulse and others, determine a specific effect on an organ or more in particular on a tissue thereof under study, which allows to see certain features rather than others.

In other words, based on which information needs to be acquired, different predefined radio frequency pulse sequences are used.

The application of said specifically predefined radio frequency pulse sequences and the subsequent detection of the signal determined by the so-called tissue relaxation under examination, that is its return to the magnetization previous to the application of the radio frequency pulse, allow to generate optimal images of said tissue under examination.

For all these reasons, the magnetic resonance is increasingly used for example for the examination of the myocardium, in particular in association with a paramagnetic contrast medium administered to the subject to be examined.

In this case, for the late acquired images after administering the paramagnetic contrast medium, by using the so-called “Inversion Recovery” radio frequency pulse sequences, indicated as late images, it is fundamental to establish the so-called inversion time TI, that is the time between the 180° inversion radio frequency pulse—after which the net magnetization is in a direction which is opposite to the static magnetic field B0—and the subsequent 90° excitation pulse which is necessary to obtain the magnetic resonance signal in the “Inversion Recovery” radio frequency pulse sequences, which must be established to detect the relaxation signal of a tissue under examination, in particular the myocardium.

For said late images acquired after administering a paramagnetic contrast medium, and, specifically for the so-called radio frequency pulse sequences Mag-IR (“Magnitude-Inversion Recovery”) and PSIR (“Phase Sensitive Inversion Recovery”), said inversion time TI is selected so that the magnetization of the myocardium without pathology corresponds to zero in the moment of the acquisition of the signal, and therefore said tissue appears black in the acquired final image.

It is in fact well known that the kinetic of a paramagnetic contrast medium is different among different tissues, since it is determined by the histocellular composition thereof. In particular, after a certain time from the administration of a paramagnetic contrast medium, the quantity of said paramagnetic contrast medium which is present in a portion of tissue without pathology, for example a portion of myocardium without pathology, will be different with respect to the one contained in a portion of tissue with an outcoming or acute or chronical pathological process, for example myocardium with an acute or chronical or outcoming pathological process.

In particular, the elimination of said paramagnetic contrast medium through the blood cycle, that is the so-called wash-out kinetic, depends on the histocellular composition of every single tissue.

It is therefore evident that using the correct inversion time TI allows to maximize the possibility of detecting said different concentration of the paramagnetic contrast medium, showing it in the detected image, and, accordingly, distinguishing, in said image, portions with different histocellular compositions, such as for example portions of tissue without pathology with respect to portions of tissue which presents evidence of an acute or chronical or pathological or outcoming pathologic process. In particular, the resonance images are acquired in a time interval in which the contrast medium, which is paramagnetic and can be therefore easily seen by resonance, is at level so as to have, in said images, the maximum difference between a tissue without pathology and a tissue with an acute or chronical or outcoming pathological process.

Conversely, using an incorrect inversion time TI, for example too long or too short, implies a reduction in the ability of distinguishing tissues with different histocellular compositions in the detected images, thus reducing, or even invalidating, the validity of the examination carried out by using the magnetic resonance apparatus.

Until today, said inversion time TI for acquiring late images after administering a paramagnetic contrast medium with “Inversion Recovery” radio frequency pulse sequences was estimated by the operator who carries out the magnetic resonance, based on its experience and generally by “trial-and-error”, which is obviously a non-optimal situation, which requires adequately trained personnel and is in particular time-consuming, the quality of the images obtained at the end of the examination with the inversion time considered by the operator as optimal being anyway not guaranteed.

From the U.S. patent publication No. US 2020/237253A1, a radio frequency pulse sequence for a magnetic resonance apparatus is known, which allows the acquisition of different images, in particular of the hearth of a subject to be examined, which uses different inversion times TI, indicated as TI scout sequence, for the test, leaving to an operator the selection of the final value for the inversion time TI to be used for the examination after a visual assessment of the series of acquired images with the different times of the TI scout sequence.

Also said known solution is anyway non-optimal and requests adequately trained personnel, since it is still time-consuming and it does not guarantee that the selection of the operator allows acquiring images of the desired quality. Furthermore, said solution requires that the subject to be examined performs an apnea that can last even 20-22 seconds, thereby causing potential inconveniences and difficulties to said subject.

It should be noted that the quality of the acquired images is fundamental. In particular, when using a non-optimal inversion time TI, the late images obtained with “Inversion Recovery” radio frequency pulse sequences after administering a paramagnetic contrast medium, which are the most important images characterizing the tissues of a subject to be examined, can be of a quality such that they do not support a possible diagnosis or even lead to an inaccurate diagnosis.

In addition to the delay that an incorrect result of this type of examination entails, the related costs and, even more particularly, the inconveniences for the subject to be examined, who could have to repeat in some cases the examination, and therefore needing to take a further quantity of paramagnetic contrast medium, which could also not be possible in some particular conditions of said subject, should be taken into account.

The technical problem of the present invention is to provide a method for determining the optimal inversion time for an “Inversion Recovery” radio frequency pulse sequence to be used in a magnetic resonance for acquiring late images of an area of interest of a subject to be examined after administering a paramagnetic contrast medium, having structural and functional features so as to automatically calculate an inversion time which is able to allow acquiring images where portions of the area of interest with different histocellular compositions are visually distinct starting from significant data of the subject to be examined and specific parameters of the carried-out examination, thereby overcoming the limitations and drawbacks which still afflict the methods made according to the prior art.

### DISCLOSURE OF INVENTION

The solution idea underlying the present invention is to determine an optimal inversion time value starting from the significant data of the subject to be examined and from specific parameters of the carried-out examination thanks to a machine learning model of an AI engine trained with values coming from a plurality of sample assessments, said optimal inversion time being adapted to be provided to a magnetic resonance apparatus for acquiring, with “Inversion Recovery” radio frequency pulse sequences, late images of an area of interest of the subject to be examined after administering a paramagnetic contrast medium where portions with different histocellular compositions are visibly distinct.

Based on this solution idea, the technical problem is solved by a method for determining an optimal inversion time for an “Inversion Recovery” radio frequency pulse sequence of a magnetic resonance of late images after administering a paramagnetic contrast medium which are acquired by a control and image capture device associated with a magnet structure of a magnetic resonance apparatus and connected to a computer adapted to exchange information and instructions with the control and image capture device, the method comprising a calculation sequence including:
a first step wherein significant data of a subject to be examined and specific parameters of an examination carried out by the magnetic resonance apparatus are collected and provided as input data to the computer;
a second step wherein the computer calculates an optimal inversion time value for the subject to be examined and for the carried-out examination by means of a machine learning model using the input data; and
a third step wherein the optimal inversion time is provided to the control and image capture device so as to drive an acquisition of resulting late images of an area of interest which are optimized for the subject to be examined and for the carried-out examination,
the machine learning model used during the second step for calculating the optimal inversion time value being optimized in a training sequence starting from a plurality of pre-calculated optimal inversion time values correlated to significant data of a related examined subject and specific parameters of the carried-out examination as established in the course of a plurality of sample assessments.

More in particular, the invention comprises the following additional and optional features, taken singularly or in combination, if necessary.

According to an aspect of the invention, the training sequence includes:
a first step of acquisition of a plurality of pre-calculated optimal inversion time values correlated to significant data of a related examined subject and specific parameters of the carried-out examination established in the course of a plurality of sample assessments;
a second step of optimization of the machine learning model based on the pre-calculated optimal inversion time values correlated to significant data of a related examined subject and specific parameters of the carried-out examination of the plurality of sample assessments; and
a third step of generation of the machine learning model so optimized.

According to another aspect of the invention, the second step of optimization uses a Random Forest based regression model as machine learning model and determines optimized values of hyperparameters thereof.

More in particular, the second step of optimization uses evolutionary computing techniques for determining the optimized values of the hyperparameters of the Random Forest based regression model.

According to another aspect of the invention, the optimal inversion time is constantly updated during an examination carried out by the magnetic resonance apparatus.

Furthermore, according to an aspect of the invention, the input data are provided to the computer through an automatic transmission of the wired or wireless type or through a manual entry, preferably by using an interface of the computer.

According to another aspect of the invention, the first data are provided to the magnetic resonance apparatus through an automatic transmission of the wired or wireless type or through a manual entry, preferably by using an interface of the magnetic resonance apparatus.

In particular, the significant data of the subject to be examined comprise anthropomorphic data, including height, weight, age of the subject and clinical data, including a medical history or any medications administered to the subject.

Further, the specific parameters of the carried-out examination comprise type of paramagnetic contrast medium as used, quantities administered, timing of administration and specifications of the resonance apparatus.

According to another aspect of the invention, the resulting late images include portions of tissue of the area of interest with several clearly distinct histocellular compositions.

Preferably, the area of interest is the myocardium of the subject to be examined.

The technical problem is also solved by a magnetic resonance apparatus provided with at least one magnet structure controlled by a control and image capture device and with a computer connected to the control and image capture device associated with calculation means, wherein the computer is adapted to implement a method for determining the optimal inversion time for an “Inversion Recovery” radio frequency pulse sequence of a magnetic resonance for acquiring late images after administration of a paramagnetic contrast medium as described above, said optimal inversion time being provided to the control and image capture device to drive an acquisition of resulting late images being optimized for a subject to be examined and for an examination carried out by the magnetic resonance apparatus.

The features and advantages of the method and apparatus according to the invention will become apparent from the description, made herein in the following, of an embodiment thereof given by way of an indicative and non-limiting example with reference to the attached drawings.

### BRIEF DESCRIPTION OF DRAWINGS

In said drawings:

FIG. 1: schematically shows a sectional view of a magnetic resonance apparatus, which uses an inversion time value determined by the method for determining the optimal inversion time for an “Inversion Recovery” radio frequency pulse sequence of a magnetic resonance for acquiring late images of an area of interest of a subject to be examined after administering a paramagnetic contrast medium according to the present invention;

FIG. 2A: schematically shows a calculation sequence of the optimal inversion time of the method according to the present invention; and

FIG. 2B: schematically shows a training sequence of the method according to the present invention.

### MODES FOR CARRYING OUT THE INVENTION

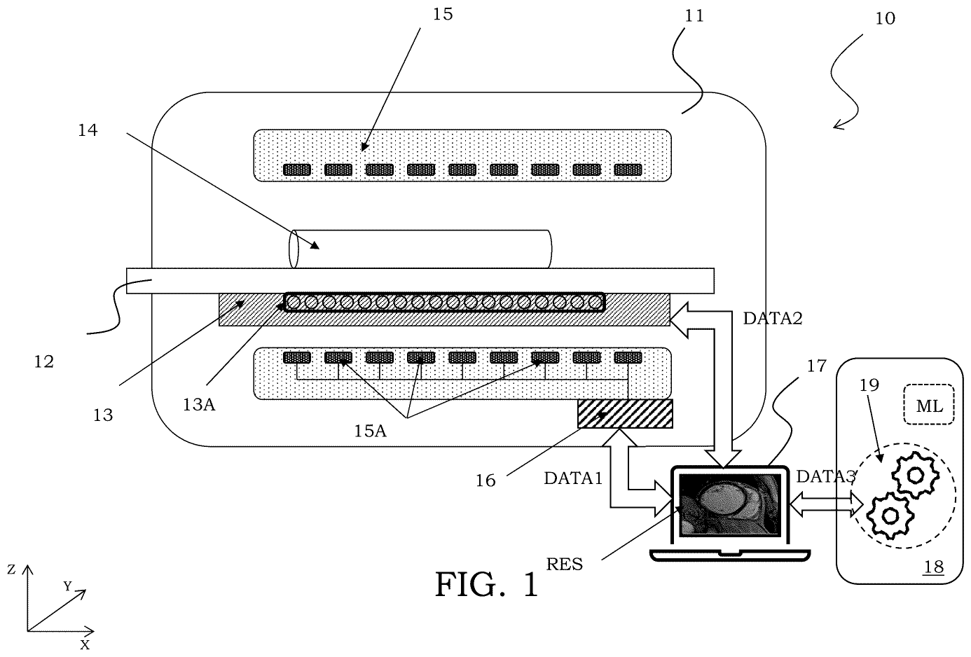

With reference to said figures, and in particular to FIG. 1, a magnetic resonance apparatus is globally indicated with 10.

Said magnetic resonance apparatus 10 essentially comprises a tubular body 11 in which a table 12 is arranged which is suitably moved by movement means 13, for example with rolls 13A, and is adapted to support a subject to be examined 14.

The magnetic resonance apparatus 10 comprises a magnet structure 15 which is suitably driven by a control and image capture device 16, which is in turn connected to a computer 17.

More in particular, the magnet structure 15 comprises a plurality of magnetic coils 15A which are connected to the control and image capture device 16 and receives therefrom suitable control signals for generating a radio frequency pulse sequence which are superimposed to a static magnetic field BO generated by the tubular body 11. As explained in relation to the prior art, the radio frequency pulse sequence is selected based on the effects which it is able to produce on an area of interest of the subject to be examined 14, in a preferred example the heart tissues.

Suitably according to the present invention, the control and image capture device 16 furthermore receives, from the computer 17, a value for an optimal inversion time TI for the “Inversion Recovery” radio frequency pulse sequence which is able to ensure the acquisition of images wherein, in the area of interest of the subject to be examined 14, portions of tissue with different histocellular composition are distinct from each other, said images being late images, that is, acquired after administering a paramagnetic contrast medium.

As seen in relation to the prior art, the inversion time TI is the time between the 180° inversion radio frequency pulse—after which the net magnetization is in an opposite direction to the static magnetic field B0—and the subsequent 90° excitation pulse which is necessary to obtain the magnetic resonance signal in the “Inversion Recovery” radio frequency pulse sequences of a magnetic resonance, which must be established in order to obtain optimal images of said area of interest in which portions with different histocellular composition are visibly distinct from each other.

The computer 17 is able to exchange information and instructions, in general terms, first data DATA1 with the control and image capture device 16 and second data DATA2 with the movement means 13.

Suitably, the computer 17 is connected to calculation means 18 provided with an AI engine 19 based on a machine learning model ML with which said computer 17 exchanges third data DATA3.

As will be explained in greater detail in the continuation of the description, the machine learning model ML of the AI engine 19 is build based on optimal inversion time values correlated to significant data of the subject to be examined and specific parameters of the carried-out examination which come from a plurality of sample assessments during a step of training of the method according to the present invention. More in particular, said computer 17 together with the calculation means 18 and the AI engine 19 implements a method for determining the optimal inversion time for an “Inversion Recovery” radio frequency pulse sequence to be used in a magnetic resonance for acquiring late images of an area of interest, in particular of the myocardium, of a subject to be examined after taking a paramagnetic contrast medium according to the present invention and provides a value of said optimal inversion time to the magnetic resonance apparatus 10, in particular to the control and image capture device 16 connected to the magnet structure 15 thereof.

Among the significant data of the subject to be examined, anthropomorphic data such as height, weight, age of the subject, as well as other clinical data such as medical history or any administered medications, can be considered, whereas the specific parameters of the carried-out examination can comprise the type of paramagnetic contrast medium as used, quantities administered, timing of administrations, specifications of the resonance apparatus which carries out the examination.

The optimal inversion time value is the value which allows to obtain significant resulting images RES, that is images in which portions of tissue of the area of interest with different histocellular compositions are clearly distinct. Suitably according to the present invention, said optimal inversion time value is calculated thanks to the machine learning model ML, in particular a Random Forest based regression model optimized starting from a plurality of optimal inversion time values correlated to significant data of the subject to be examined and specific parameters of the carried-out examination which come from a plurality of sample assessments.

The method for determining the optimal inversion time for an “Inversion Recovery” radio frequency pulse sequence for acquiring late images after administrating a paramagnetic contrast medium by means of magnetic resonance according to the present invention is used for acquiring heart images after administering a paramagnetic contrast medium to a subject to be examined, and originates from the fact that, after the administration of said paramagnetic contrast medium, the heart wash-out curve depends on significant data of the subject to be examined, in particular personal and clinical data, but also specific parameters of the carried-out examination, in particular the paramagnetic contrast medium which is used and the magnet structure of the magnetic resonance apparatus which carries out the examination.

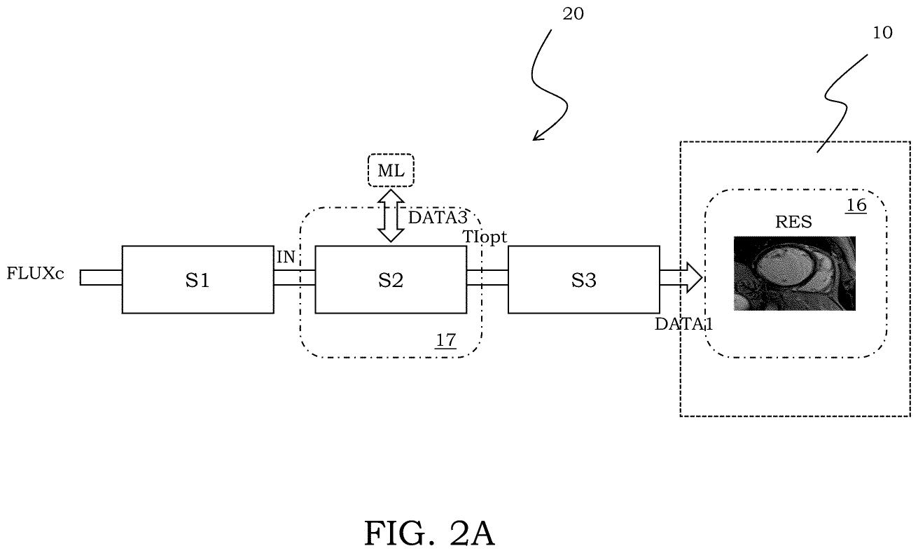

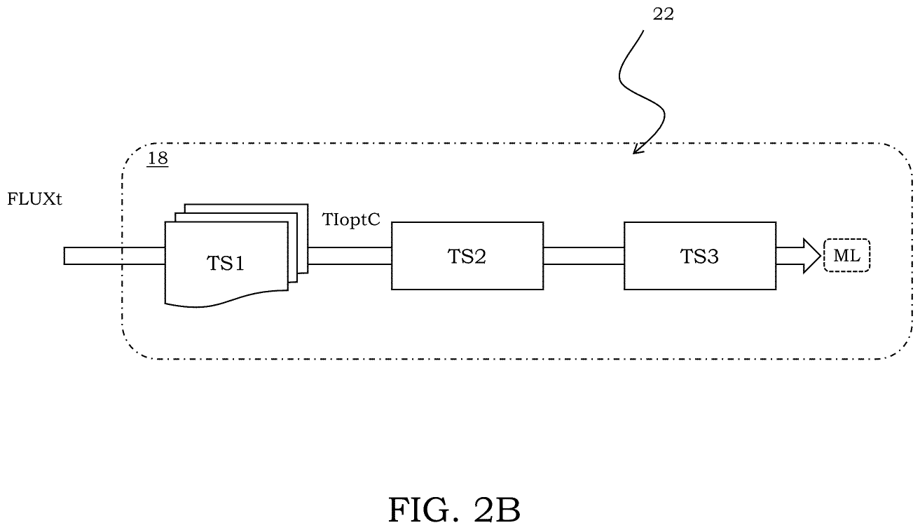

According to the present invention, the method therefore essentially comprises a calculation sequence 20, including a plurality of steps FLUXc, as schematically illustrated in FIG. 2A. In particular, the calculation sequence 20 comprises:
a first step S1 wherein significant data of the subject to be examined and specific parameters of the carried-out examination are collected and provided as input data IN to the computer 17 associated with the magnetic resonance apparatus 10;
a second step S2 wherein the computer 17 uses a machine learning model ML for calculating an optimal inversion time value TIopt for a specific subject to be examined and for a specific examination to be carried out, that is using the input data IN; and
a third step S3 wherein said optimal inversion time TIopt is provided as first data DATA1 to the magnetic resonance apparatus 10 and in particular to the control and image capture device 16 thereof so as to allow the acquisition of resulting late images RES optimized for the specific subject to be examined and for the specific carried-out examination.

The input data IN can be provided to the computer 17 through an automatic transmission of the wired or wireless type or through a manual entry, by using for example an interface of said computer 17 which is suitably arranged for the purpose. Analogously, the first data DATA1 can be provided to the magnetic resonance apparatus 10 and in particular to the control and image capture device 16 thereof through an automatic transmission of the wired or wireless type or through a manual entry by using for example an interface of said magnetic resonance apparatus 10.

Suitably according to the present invention, the optimal inversion time value TIopt is constantly updated through the calculation means 18 during the examination, as the time passes from the administration of the paramagnetic contrast medium up to the end of said examination.

In a preferred embodiment, the second step S2 uses a Random Forest based regression model as machine learning model ML.

As it is well known, the Random Forest model is suitably optimized by identifying the optimal values for the so-called hyperparameters which partially modify the behavior/operation and “what to give importance to” during the training in a sort of decision tree, said hyperparameters being established through optimization techniques, in particular through evolutionary computing.

The evolutionary computing is substantially a calculation process in which a plurality of hyperparameter combinations is tested and a subset of combinations, identified as the best ones, is selected, subsequently generating other combinations starting from the selected ones and iterating the entire process up to the identification of the optimal values of the hyperparameters of said Random Forest model.

Suitably according to the present invention, the machine learning model ML is optimized in a learning sequence 22 of the method according to the present invention, which is schematically illustrated in FIG. 2B, starting from pre-calculated optimal inversion time values and from corresponding significant data of the subject to be examined and specific parameters of the carried-out examination as established in the course of a plurality of sample assessments carried out by specialized personnel.

The training sequence 22 comprises a plurality of steps FLUXt, particularly including:
a first step TS1 of acquisition of a plurality of pre-calculated optimal inversion time values TIoptC correlated to significant data of a related examined subject and specific parameters of the carried-out examination established in the course of a plurality of sample assessments;
a second step TS2 of optimization of a machine learning model ML based on said pre-calculated optimal inversion time values TIoptC correlated to significant data of a related examined subject and specific parameters of the carried-out examination of the plurality of sample assessments; and
a third step TS3 of generation of said machine learning model ML so optimized.

Suitably, using a Random Forest based regression model as machine learning model ML, the second step TS2 of optimization establishes the values of the hyperparameters thereof, in particular optimized through evolutionary computing techniques.

The so-optimized machine learning model ML is then used by the calculation means 18 of the computer, and in particular by the AI engine 19, for calculating the optimal inversion time value TIopt for a specific subject to be examined and for a specific examination to be carried out starting from the input data IN.

In other words, the method for determining the optimal inversion time for an “Inversion Recovery” radio frequency pulse sequence for acquiring late images through magnetic resonance after administration of a paramagnetic contrast medium according to the present invention receives, as input, the significant data of the subject to be examined and the specific parameters of the carried-out examination, which affect the kinetic of the paramagnetic contrast medium, in addition to the time passed since the administration of the paramagnetic contrast medium and, based on said data, provides an optimal inversion time TIopt, which is customized on the profile of the subject to be examined and of the examination to be carried out, and is to be used for acquiring late images, which are of a high quality and diagnostically significant, by means of the magnetic resonance apparatus 10.

Clinical tests show that the method for determining the optimal inversion time for an “Inversion Recovery” radio frequency pulse sequence of a magnetic resonance according to the present invention is able to calculate the optimal inversion time values with an accuracy comparable to that of a skilled operator. Therefore, the use of said method reduces the operator-dependency of the magnetic resonance diagnostics as a whole.

Suitably, the method according to the present invention therefore reduces the number of images which must be acquired several times to optimize the quality, that is the contrast between portions with different histocellular composition, and keeps unchanged, and possibly reduces, the overall duration of the examination.

Obviously, a person skilled in the art can make various modifications and variations to the method described above, in order to satisfy contingent and specific needs, all being included in the scope of protection of the invention as defined by the following claims.

1-12. (canceled)

13. A method for determining an optimal inversion time for an “Inversion Recovery” radio frequency pulse sequence of a magnetic resonance for acquiring late images after administering a paramagnetic contrast medium by a control and image capture device associated with a magnet structure of an apparatus for magnetic resonance and connected to a computer configured to exchange information and instructions with the control and image capture device, the method comprising:
performing a training sequence including:
optimizing a machine learning model starting from a plurality of calculated optimal inversion time values correlated to significant data of a related examined subject and specific parameters of a carried-out examination as established in the course of a plurality of sample assessments; and
performing a calculation sequence including:
a first step wherein significant data of a subject to be examined and specific parameters of an examination carried out by the magnetic resonance apparatus are collected and provided as input data to the computer;
a second step wherein the computer calculates an optimal inversion time value for the subject to be examined and for the carried-out examination by a machine learning model using the input data; and
a third step wherein the optimal inversion time is provided to the control and image capture device so as to drive an acquisition of resulting late images of an area of interest which are optimized for the subject to be examined and for the carried-out examination,
wherein the second step of the calculation sequence uses at least in part the machine learning model as optimized in the training sequence for calculating the optimal inversion time value.

14. The method according to claim 13, wherein the training sequence further includes:
a first step of acquisition of a plurality of calculated optimal inversion time values correlated to significant data of a related examined subject and specific parameters of the carried-out examination established in the course of a plurality of sample assessments;
a second step of optimization of the machine learning model based on the calculated optimal inversion time values correlated to significant data of a related examined subject and specific parameters of the carried-out examination of the plurality of sample assessments; and
a third step of generation of the machine learning model so optimized.

15. The method according to claim 14, wherein the second step of optimization of the training sequence uses at least on part a Random Forest based regression model as machine learning model and determines optimized values of hyperparameters thereof.

16. The method according to claim 15, wherein the second step of optimization of the training sequence uses at least in part evolutionary computing techniques for determining the optimized values of the hyperparameters of the Random Forest based regression model.

17. The method according to claim 13, wherein the optimal inversion time is constantly updated during an examination carried out by the magnetic resonance apparatus.

18. The method according to claim 13, wherein the input data are provided to the computer through an automatic transmission selected from a group consisting of: an automatic transmission of the wired type, an automatic transmission of the wireless type, and a manual entry.

19. The method according to claim 13, wherein the first data are provided to the magnetic resonance apparatus through a method selected from a group consisting of: an automatic transmission of the wired type, an automatic transmission of the wireless type, and a manual entry.

20. The method according to claim 13, wherein the significant data of the subject to be examined is selected from a group consisting of: anthropomorphic data, including height, weight, age of the subject, and clinical data, including a medical history or any medications administered to the subject.

21. The method according to claim 13, wherein the specific parameters of the carried-out examination is selected from a group consisting of: type of paramagnetic contrast medium as used, quantities administered, and timing of administration and specifications of the resonance apparatus.

22. The method according to claim 13, wherein the resulting late images include portions of tissue of the area of interest with several clearly distinct histocellular compositions.

23. The method according to claim 13, wherein the area of interest is the myocardium of the subject to be examined.

24. A magnetic resonance apparatus provided with at least one magnet structure controlled by a control and image capture device and with a computer connected to the control and image capture device associated with calculation means, wherein the computer is configured to implement a method for determining the optimal inversion time for an “Inversion Recovery” radio frequency pulse sequence of a magnetic resonance for acquiring late images after administration of a paramagnetic contrast medium, comprising:
a training sequence including:
optimizing a machine learning model starting from a plurality of calculated optimal inversion time values correlated to significant data of a related examined subject and specific parameters of a carried-out examination as established in the course of a plurality of sample assessments; and
a calculation sequence including:
a first step wherein significant data of a subject to be examined and specific parameters of an examination carried out by the magnetic resonance apparatus are collected and provided as input data to the computer;
a second step wherein the computer calculates an optimal inversion time value for the subject to be examined and for the carried-out examination by a machine learning model using the input data; and
a third step wherein the optimal inversion time is provided to the control and image capture device so as to drive an acquisition of resulting late images of an area of interest which are optimized for the subject to be examined and for the carried-out examination,
the second step of the calculation sequence using at least in part the machine learning model as optimized in the training sequence for calculating the optimal inversion time value.
wherein the optimal inversion time is provided to the control and image capture device to drive an acquisition of resulting late images being optimized for a subject to be examined and for an examination carried out by the magnetic resonance apparatus.

25. The magnetic resonance apparatus according to claim 24, wherein the training sequence includes:
a first step of acquisition of a plurality of calculated optimal inversion time values correlated to significant data of a related examined subject and specific parameters of the carried-out examination established in the course of a plurality of sample assessments;
a second step of optimization of the machine learning model based on the calculated optimal inversion time values correlated to significant data of a related examined subject and specific parameters of the carried-out examination of the plurality of sample assessments; and
a third step of generation of the machine learning model so optimized.

26. The magnetic resonance apparatus according to claim 24, wherein the optimal inversion time is constantly updated during an examination carried out by the magnetic resonance apparatus.

27. The magnetic resonance apparatus according to claim 24, wherein the input data are provided to the computer through a method selected from a group consisting of: an automatic transmission of the wired type, an automatic transmission of the wireless type, and a manual entry.

28. The magnetic resonance apparatus according to claim 24, wherein the resulting late images include portions of tissue of the area of interest with several clearly distinct histocellular compositions.

29. The magnetic resonance apparatus according to claim 24, wherein the area of interest is the myocardium of the subject to be examined.

30. The method according to claim 18, wherein the input data are provided to the computer using an interface of the computer.

31. The method according to claim 19, wherein the first data are provided to the magnetic resonance apparatus using an interface of the magnetic resonance apparatus.

32. A method for determining an optimal inversion time for an “Inversion Recovery” radio frequency pulse sequence of a magnetic resonance for acquiring late images after administering a paramagnetic contrast medium by a control and image capture device associated with a magnet structure of an apparatus for magnetic resonance and connected to a computer configured to exchange information and instructions with the control and image capture device comprising:
performing a training sequence including:
a first step of acquisition of a plurality of calculated optimal inversion time values correlated to significant data of a related examined subject and specific parameters of a carried-out examination established in the course of a plurality of sample assessments;
a second step of optimization of a machine learning model based on the calculated optimal inversion time values correlated to significant data of a related examined subject and specific parameters of the carried-out examination of the plurality of sample assessments; and
a third step of generation of the machine learning model so optimized; and
performing a calculation sequence including:
a first step wherein significant data of a subject to be examined and specific parameters of an examination carried out by the magnetic resonance apparatus are collected and provided as input data to the computer;
a second step wherein the computer calculates an optimal inversion time value for the subject to be examined and for the carried-out examination by a machine learning model using the input data; and
a third step wherein the optimal inversion time is provided to the control and image capture device so as to drive an acquisition of resulting late images of an area of interest which are optimized for the subject to be examined and for the carried-out examination,
wherein the second step of the calculation sequence uses at least in part the machine learning model as optimized in the training sequence for calculating the optimal inversion time value.
